(19) United States
(12) Patent Application Publication
(10) Pub. No.: US 2011/0160814 A2
(43) Pub. Date: Jun. 30, 2011

(54) PHOTOTHERAPY APPARATUS FOR HAIR, SCALP AND SKIN TREATMENT

(75) Inventors: Gavin Tucker, Irvine, CA (US); Nicholas Brox, Laguna Beach, CA (US); Jeffrey Braile, Boca Raton, FL (US); Morgan Pepitone, Newport Beach, CA (US)

(73) Assignee: APIRA Science, Inc., Newport Beach, CA (US)

(21) Appl. No.: 12/586,290

(22) Filed: Sep. 18, 2009

(51) Int. Cl. A61N 5/06

(52) U.S. Cl. 607/90

(57) ABSTRACT

A wearable hands-free apparatus for providing phototherapy treatment to a number of hair, scalp and skin related conditions includes a head unit (e.g., a headset, headphones, headband, or helmet unit) with earphones to allow the user to listen to an audio program during a treatment. The head unit supports a light emitting canopy band or plate fitted with an array of light generating sources, such as light emitting diodes (LEDs), laser diodes, infrared lights or other light sources, that emit light within a particular wavelength range correlating with the treatment of one or more specific hair, scalp and/or skin-related conditions. The light emitting canopy band or plate is specifically designed to conform to the shape of the human scalp for providing complete light coverage to the areas of the scalp that are most commonly affected by hair loss in men and women. In a further embodiment, a fixed or detachable face plate connects to the head unit and houses an array of the light generating sources for treating various facial skin-related conditions. A control system, preferably in a handheld device, allows the user to select the desired treatment program and audio operations.

This application is based on two U.S. provisional patent applications: Ser. No. 61/136,630 filed on Sep. 19, 2008; and Ser. No. 61/211,630 filed on Apr. 1, 2009.

### BACKGROUND OF THE INVENTION

1. Field of the Invention

This invention relates to skin, scalp and hair treatment and, more particularly, to a device that combines light generating sources with a headset, headphones, headband, or helmet unit, capable of providing therapeutic aid to a user's skin, scalp and/or hair by way of evenly distributed light of various wavelength directed onto selected areas of a user's skin or scalp tissue.

2. Discussion of the Related Art

People are frequently confronted with hair loss as well as a variety of different scalp and skin-related conditions, such as acne, sun spots, and wrinkling of the skin, psoriasis and non-melanoma skin cancer. In response, an assortment of treatment products, each typically targeting one specific hair, scalp or skin-related condition, has been developed and made available to the public. Many of these products are in the form of a topical solution that requires an arduous application process. Where the condition is hair loss, a surgical process has been made available, wherein hair plugs are surgically transplanted in place of the missing hair. However, this surgical process for treating hair loss is extremely expensive, and consequently, not available to an average consumer.

This invention pertains to the field of Phototherapy. Phototherapy consists of exposure to specific wavelengths of light using lasers, light emitting diodes (LED's) (both individual and arrays), IPL's (Intense Pulsed Light) and other light sources, for a prescribed amount of time to both treat disease and affect cosmetic enhancements to the hair, scalp and skin. The use of phototherapy in medical science and aesthetics is rapidly evolving as more and more wavelengths of light are being identified to target various sections of cells in order to stimulate cellular proficiency and enhance the body's ability to heal and rejuvenate itself. Phototherapy is currently used to treat acne, wrinkles, sun and age spots, rosacia, eczema, hair loss and wound healing through wavelengths indicated by various colors (i.e., wavelengths) of the light spectrum. By utilizing various wavelengths, colors relatively close on the spectrum can cause different effects when applied to various parts on the body.

For example, red light at 670 nanometers has been clinically shown to prevent hair loss and re-grow new hair, as well as to cause increased melanin production and protein synthesis. Red and infrared lights have also been used to increase the production of collagen and to reduce redness, dilated capillaries and damage to the skin as well as reduction of wrinkles and fine lines. Blue light has been clinically shown to reduce acne and, when combined with red light, eliminates acne and reduces the scarring often associated with acne treatment. Yellow and Amber lights have been clinically shown to reduce fine lines and wrinkles, rosacia, and can help to repair sun damaged skin. Green light has been shown to reduce and eliminate sun and age spots, lighten freckles and also help promote more luminous skin condition and overall radiance of the skin. As set forth above, many of these light sources have multiple benefits, cross over each other in treating certain ailments and work to promote a variety of benefits to the hair and skin. These light sources are often used in combinations to provide increase efficacy and various degrees of stimulation.

The present invention provides for the application of phototherapy to the face and scalp utilizing a delivery system designed to maximize the effectiveness of all of these treatments, either singly or in combination, through use of either fixed or removable bands or plates of light. The present invention utilizes a technique to enable the user to have a phototherapy device that does not require any manual movements of the light sources and which further enables the user to have the device suspended from the top of the head via a set of headphones, headband or helmet, placing a large quantity of the light sources in close proximity to either the face or scalp. This provides the user with a fixed light source that enables absorption of the light during a timed period.

Science throughout the years has determined the effects of various wavelengths of light, but absorption is the key to cellular change. Light therapy emits photons which are absorbed by the skins photoreceptors. Hair and skin cells respond well to phototherapy involving low level light due to the fact that cells reside just underneath the skin surface making these low levels of energy able to reach the receptor sites and induce photochemistry.

There are a number of phototherapy devices currently available for home use to treat both skin and hair. The majority of these are hand held devices, varying in both size and number of light sources (i.e., laser diodes, LED's, or infrared diodes). These devices are manually moved around the hair or face by the user and require a constant movement in order to expose the entire surface area to the light sources. This provides for an uneven treatment protocol, as the average user is unlikely to be able to cover the entire surface area through manual movements and will leave certain areas untreated. Further, due to the need for a manageable size (must fit in the hand), these devices are often underpowered as it is difficult to fit an adequate number of light sources in the hand held unit.

Only a few phototherapy devices have been developed that are adapted to be portably worn by a user in a hands-free mode of operation. For example, U.S. Pat. App. Pub. No. 2009/0012586 A1 discloses a system that houses LEDs within a hat, targeting a reduction in hair loss and the therapeutic healing of a variety of skin disorders. However, this device does not treat skin-related conditions on the face, and it further requires the onerous activity of snapping or screwing in different LEDs to alter the desired wavelength.

U.S. Pat. App. Pub. No. 2006/0030908 discloses a skin treatment phototherapy device that may comprise a clamshell structure, pen shape, facial mask, or desk lamp design, which includes multi-colored LEDs. The device attempts to treat a variety of skin conditions on the face and other skin regions below the user's head. Depending on the skin condition to be treated, the corresponding wavelengths, intensity levels, and time interval for the skin treatment are received by a control system and subsequently emitted by the device's multi-colored LEDs. However, this device is neither designed nor intended to treat hair loss, and it further lacks an ideal embodiment for supplying an evenly distributed light pattern upon a user's face.

While these hair treatment devices and skin treatment devices, all using LED technology, are minimally useful for their intended purposes, there remains a need for a hands-free phototherapy apparatus that offers better uniformity of light distribution and intensity with enhanced penetration depth control. There is a further need for a hands-free phototherapy apparatus that provides interchangeable light emitting canopy bands or face plates for treating a variety of skin-related conditions and hair loss, wherein each band or plate has an arrangement of lights of a particular wavelength range that is useful for treatment of one or more skin, scalp or hair related conditions.

The present invention seeks to address the limitations and shortcomings of presently known phototherapy treatment devices, as well as to eliminate the potential for human error, by creating a singular band or plate, clustered with either laser diodes, LED's (both individual and arrays), IPL's or other light sources which is either suspended right above the scalp or directly in front of the face through an attachment to a set of headphones, headband or helmet. This band is able to contain an adequate number of light sources as it covers the entire treatment area. It can be equipped with a variety of different light source output levels whether 5 mw, 20 mw or 100 mw. The user simply sets the timed operation on a control (e.g., a handheld device), by selecting a desired hair, scalp or skin treatment to activate a timed treatment according to a particular treatment protocol, and the automated session begins.

### SUMMARY OF THE INVENTION

The present invention is directed to a wearable hands-free apparatus that provides phototherapy treatment to the scalp, skin tissue, and layers of a user's dermis. The phototherapy apparatus utilizes an array of light generating sources, which are housed within a unique canopy band or face plate, structured to provide evenly distributed light to the user's skin or scalp. For this application, the phrase “light generating sources” includes, but is not limited to, light emitting diodes (LEDs), laser diodes, infrared, and intense pulse lights (IPLs). This photo-biostimulation process produces an increase in ATP and keratin production, enhancement in blood flow and circulation, as well as an increase in collagen production as previously noted, phototherapy can be used to treat hair loss, and a number of skin and scalp conditions, such as acne, sunspots, wrinkle reduction, skin tightening, psoriasis, eczema and collagen production.

Each form of treatment requires light emitted within a particular wavelength range in order to sufficiently be absorbed into the skin tissue to treat a user's particular skin, scalp or hair-related condition. The canopy band or plate houses an array of light generating sources that are capable of emitting light within a range of output wavelengths in order to provide one or more penetration depths and photo-biostimulation effects. In a further embodiment of the invention, each canopy band may contain an array of mixed light generating sources, wherein certain light generating sources emit light within one wavelength range, while other light generating sources emit light within different wavelength ranges, thereby targeting different areas of the cell.

In a preferred embodiment of the invention, the phototherapy apparatus is comprised of a head unit, whether it be a headset, headphones, headband, or helmet unit, that includes the canopy band or plate with the array of light generating sources for treating hair and scalp related conditions. The canopy band is specifically designed to conform to the shape of the human scalp in order to provide complete light coverage to the areas that are most commonly affected by hair loss in both men and women. The canopy band may be fixed as an integral part of the head unit or, alternatively, may be interchangeably attached by way of a releasable securing mechanism. Various embodiments of the releasable securing mechanism utilizing different methods of interchangeable attachment are contemplated. The light generating sources (e.g., diodes) may be adapted to pulse according to a proprietary algorithm that is programmed in the memory of a control device. In a preferred embodiment, the control device is a hand held unit with an LCD touch responsive control display. Various hair loss treatments and/or skin therapy protocols for both men and women can be selected on the control display. The algorithm may provide for pulsed light in specific pre-determined patterns in accordance with the particular hair loss or skin related treatment that is selected.

### BRIEF DESCRIPTION OF THE DRAWINGS

For a fuller understanding of the nature of the present invention, reference should be made to the following detailed description taken in conjunction with the accompanying drawings in which:

FIG. 1 is a side profile view showing the phototherapy apparatus of the present invention, in accordance with one embodiment, and including a canopy band or plate with an array of light generating sources emitting light within a range of wavelengths, which is positioned in spaced, opposing relation to the user's face, a headset unit, an interchangeable point for attaching or detaching canopy bands, a set of headphones, a set of controls for controlling the operations of the apparatus, an LCD timer and function display system, an input for a rechargeable battery system, and an audio input;

FIG. 2 is a side profile view showing the phototherapy apparatus of FIG. 1 and including a canopy band or plate with an array of light generating sources emitting light within a range of wavelengths, which is positioned in spaced, opposing relation to the user's scalp for treatment of hair and scalp conditions, canopy band spacing columns, a set of headphones, an interchangeable point for attaching or detaching canopy bands, a set of controls for controlling the operations of the apparatus, an LCD timer and function display system, an input for a rechargeable battery system, and an audio input;

FIG. 3 is an exploded side elevational view of the embodiment of FIG. 1 showing the phototherapy apparatus of the present invention, in accordance with a preferred embodiment, as it is when dismantled into separate parts, and including a canopy band or plate with an array of light generating sources, a set of headphones, and a head support band;

FIG. 4 is an isolated view of the male component taken from FIG. 3, including two release buttons, and a releasing mechanism;

FIG. 5 is a front view showing the phototherapy apparatus of FIG. 1, as it is when dismantled into separate parts, and including a canopy band with an array of light generating sources and headphones;

FIG. 6 is a side profile view showing the phototherapy apparatus of the present invention, in accordance with another embodiment, and including a canopy band or plate with an array of light generating sources, which is positioned in spaced, opposing relation to the user's scalp for treatment of hair and/or scalp conditions, an interchangeable point for attaching or detaching canopy bands, a headband unit, a headband securing mechanism, an LCD timer and function display system, an input for a rechargeable battery system, and an audio input;

FIG. 7 is a side profile view showing the phototherapy apparatus of the present invention, in accordance yet a further embodiment, and including a fixed or detachable face plate with an array of light generating sources, which is positioned in spaced, opposing relation to the user's face, a canopy band with an array of light generating sources emitting light within a range of wavelengths, which is positioned in spaced, opposing relation to the user's scalp for treatment of hair-related conditions, a faceplate fastening mechanism, a headset unit, an interchangeable point for attaching or detaching canopy bands, a set of headphones, a set of controls for controlling the operations of the apparatus, an LCD timer and function display system, an input for a rechargeable battery system, and an audio input;

FIG. 8 is a side profile view showing the phototherapy apparatus of the present invention, in accordance with a further embodiment, and including a fixed or detachable face plate with an array of light generating sources, which is positioned in spaced, opposing relation to the user's face, a faceplate fastening mechanism, a helmet unit, a set of controls for controlling the operations of the apparatus, an LCD timer and function display system, an input for a rechargeable battery system, and an audio input;

FIG. 9 is an isolated inner view of the canopy band or plate comprising an array of light generating sources mounted on the inner facing side of the canopy band or plate, showing the light consistency widening as it leaves the light generating source's aperture, as well as the resulting overlap of light on skin surface;

FIG. 10 is an isolated view of the inner facing side of a canopy band or plate, showing the array of light generating sources and the male component of the releasing mechanism;

FIG. 11 is a side profile view showing the phototherapy apparatus of the present invention, in accordance with a preferred embodiment, and including a canopy band or plate fitted with an array light generating sources and earphones, and wherein the canopy band is specifically designed to conform to the shape of the human scalp for providing complete light coverage to the areas on the scalp that are most commonly affected by hair loss in both men and women;

FIG. 12 is a top plan view of the canopy band of the embodiment of FIG. 11, illustrating the unique design that conforms to the shape of the human scalp to provide complete light coverage to the areas that are most commonly affected by hair loss in men and women;

FIG. 13 is a perspective view of the phototherapy apparatus of FIG. 11;

FIG. 14 shows the headphones of the phototherapy apparatus of FIG. 11 folded up under the canopy band or plate in a collapsed position for storage, packaging and/or transport; and

FIG. 15 is a top plan view of a hand held control unit that connects to the phototherapy apparatus for selecting timed phototherapy treatments according to the most common hair loss patterns or skin related conditions in both men and women, which specific illustrations of male and female baldness patterns shown on treatment selection buttons.

Like reference numerals refer to like referenced parts throughout the several views of the drawings.

### DETAILED DESCRIPTION OF THE PREFERRED EMBODIMENTS

Referring to the several views of the drawings, the wearable hands-free apparatus that provides phototherapy treatment to the scalp, skin tissue, and layers of a user's dermis is shown according to several embodiments of the invention and is generally indicated as 10. The phototherapy apparatus 10 is specifically sized, structured and configured to be worn on a person's head.

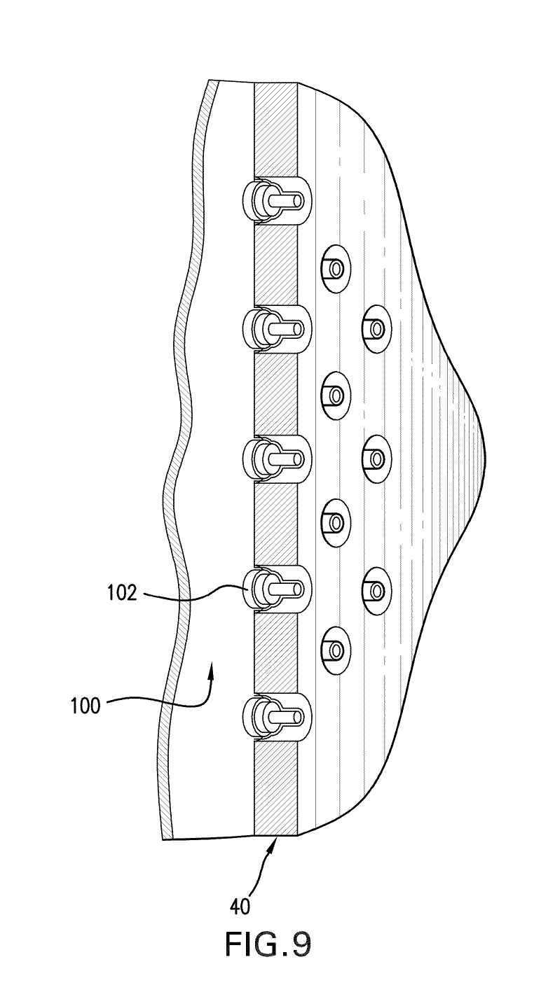

In each of the embodiments of the invention, the phototherapy apparatus 10 includes a head unit 12 (e.g., a headset, head phones, headband, or helmet) with left and right audio earphones 24 to allow the user to listen to an audio program during a phototherapy treatment. The head unit 12 supports a light emitting canopy band or plate 40 that houses an array of light generating sources 102 (see FIG. 9), such as light emitting diodes (LEDs), lasers, infrared lights, or other suitable light sources that are adapted to emit light within a particular wavelength range correlating with the treatment of one or more specific hair loss, scalp and/or skin-related conditions.

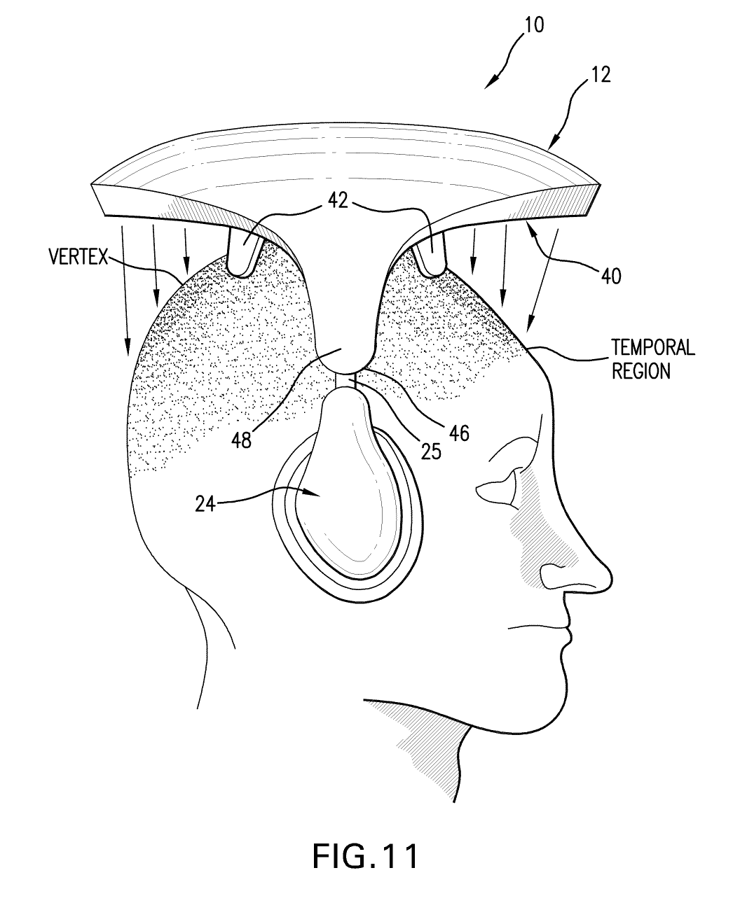

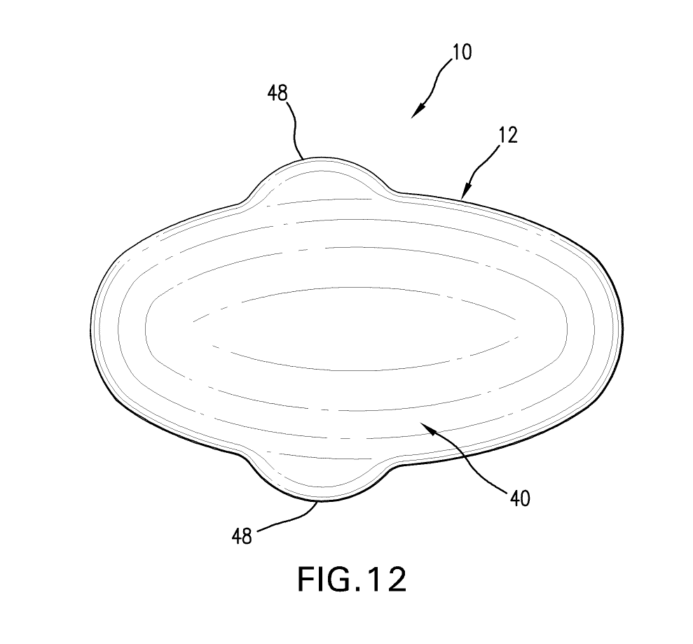

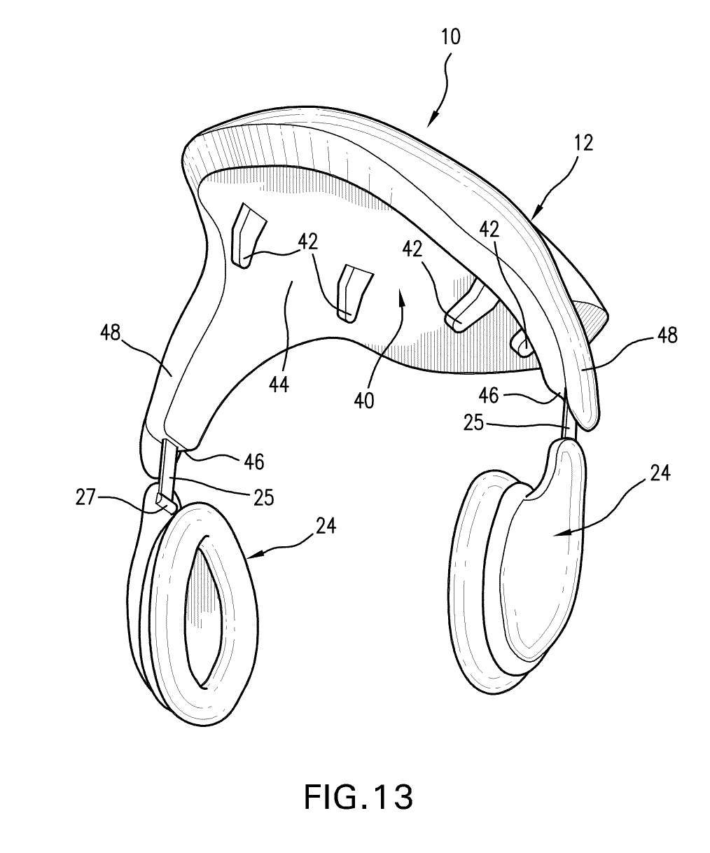

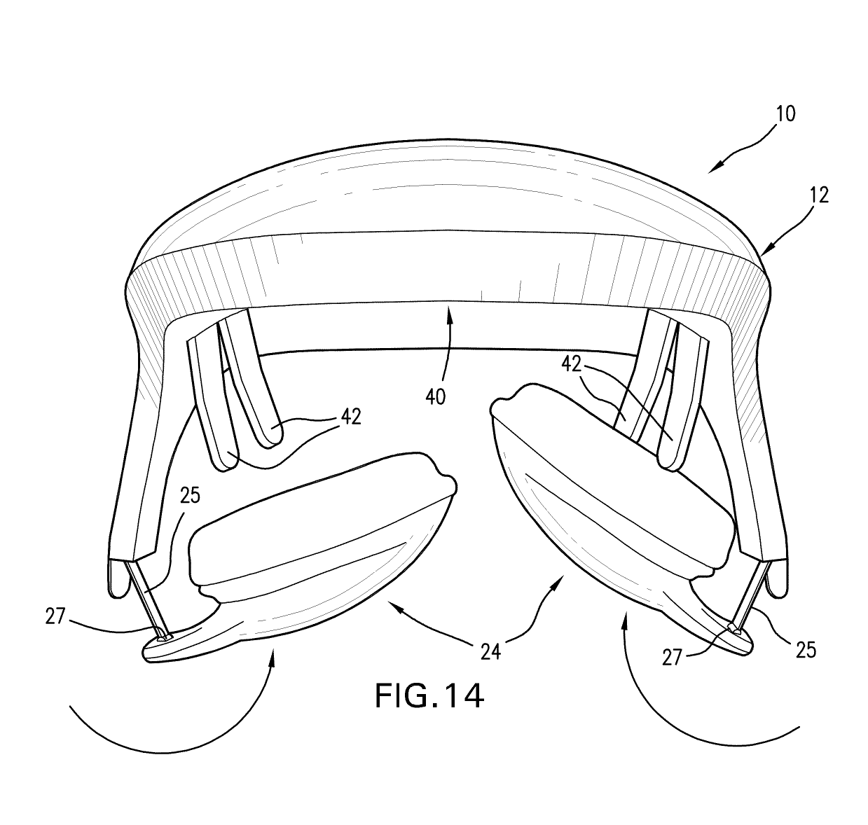

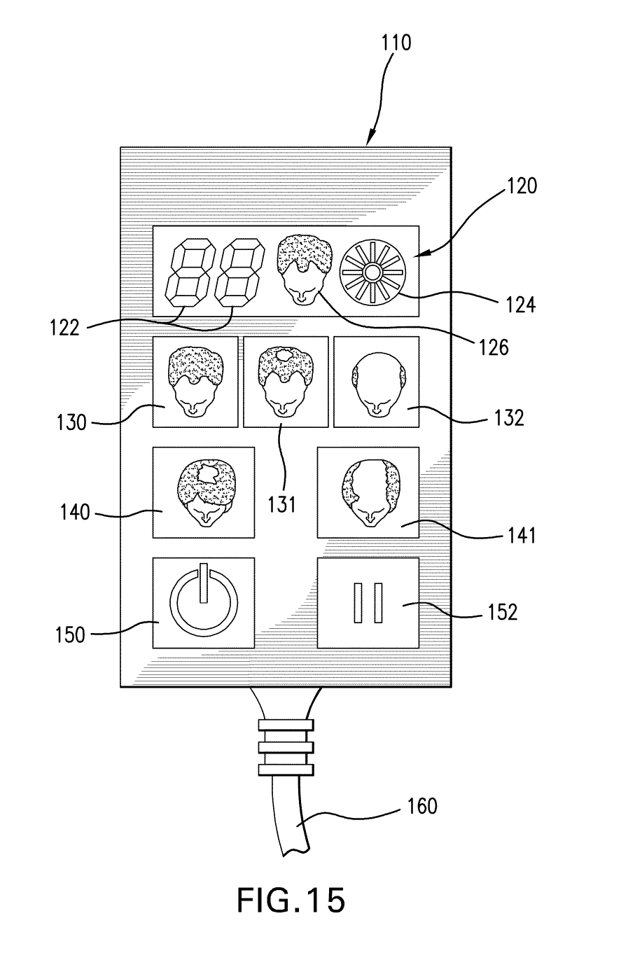

A preferred embodiment of the phototherapy apparatus 10 is shown in FIGS. 11-15. In this preferred embodiment of the invention, the canopy band or plate 40 is formed as an integral part of the head unit 12 and is specifically designed to conform to the shape of the human scalp for providing complete light coverage to the areas of the scalp that are most commonly affected by hair loss in both men and women. The canopy band or plate 40, as seen in FIGS. 11-14, is slightly elongated at the front and rear ends to emphasize the unique shaping of the human scalp. The canopy band or plate 40 is also designed with a slight taper from front to rear, to allow the light deposited on the scalp to treat the temporal region and vertex of the scalp, while covering the entire scalp for a complete phototherapy treatment. The underside surface 44 of the canopy band or plate 40, disposed in spaced, opposing relation to the user's scalp (see FIG. 11), is fitted with the light emitting sources 102 (e.g., diodes) that may be adapted to pulse according to the proprietary algorithm that is programmed in the memory of a hand held control unit 110, shown in FIG. 15. This algorithm provides for pulsed light in specific predetermined patterns in order to treat a variety of hair loss conditions in both men and women. Forward and rear spacing columns 42 extend downwardly from the underside 44 of the canopy band or plate 40. Distal ends of the spacing columns 44 engage the user's head (e.g., the scalp) to maintain a predetermined space (i.e., gap) between the array of light generating sources 102 and the user's scalp, thereby ensuring proper light distribution and penetration of light into the cells in the scalp. The left and right audio headphones 24 are adjustably supported on slidable arm members 25 that extend and retract from the head unit 12 at the bottom ends 46 of downwardly extending portions 48 on the left and right sides of the head unit 12. The left and right audio headphones 24 are also adapted to fold inwardly and under the canopy band or plate 40, as shown in FIG. 14. Specifically, hinge members 27 connecting the headphones 24 to the arm members 25 allow the headphones to fold and collapse under the canopy band or plate 40 for convenient storage, packaging and transport.

Referring to FIG. 15, the hand held control unit 110 is shown and includes an LCD display 120 with timer functions and treatment control and selection buttons. Specifically, the top LCD display 120 presents a two digit timer display 122 for indicating the number of minutes remaining in a particular phototherapy treatment. The opposite side of the top LCD display 120 presents a timer wheel 124 that counts down five second intervals of each minute. More specifically, the time wheel includes an arrangement of spokes representing five seconds in the timer display function. During operation, a spoke on the timer wheel disappears after each five seconds of operation, within a one minute cycle. For example, during a twenty minute treatment, the two digit timer display 122 will present the number 20 and, at the beginning of the treatment the timer wheel will present twelve spokes. After every five seconds, one of the spokes on the timer wheel will disappear until the minute is up. Then, the number on the two digit display will change from 20 to 19 and the timer wheel will repopulate with 12 spokes to resume the countdown sequence for each minute of treatment. The illustration in the center of the top LCD display presents an image 126 of the top of a male or female head, with a particular balding pattern representing the specific treatment that has been selected. Below the top LCD display there is a row of three buttons 130, 131 and 132, each presenting an image of the top of a male scalp with illustrations of hair loss patterns that are common in men. The next row of treatment control buttons includes two female treatment controls 140, 141 with images of the top of a female scalp presenting two common hair loss patterns in women. The control unit 110 further includes an on/off button 150 and a start/pause button 152. When the user selects a particular hair loss treatment for either a man or woman, by pressing one of the five treatment buttons, the scalp image from the selected treatment is presented in the center of the top LCD display 120, indicating that this particular treatment has been selected. The user can then press the start button 152 which will start the timed automated phototherapy treatment session. The algorithm, programmed in the memory of the control unit, may provide for pulsed light from the diodes in the canopy band, in specific predetermined patterns in accordance with the particular hair loss treatment that is selected. The hand held control unit 110 connects to the head unit 12 by a wire 160 that extends from the hand held unit 110 and plugs into the head unit 12 at a designated port. The hand held unit stores all programmed functions of the phototherapy apparatus in memory including operational functions of the array of light generating sources 102, as well as all audio functions connected with the headphones 24 on the head unit 12. The hand held control unit 110 provides for selection of audio programs stored in memory, as well as volume and other audio functions.

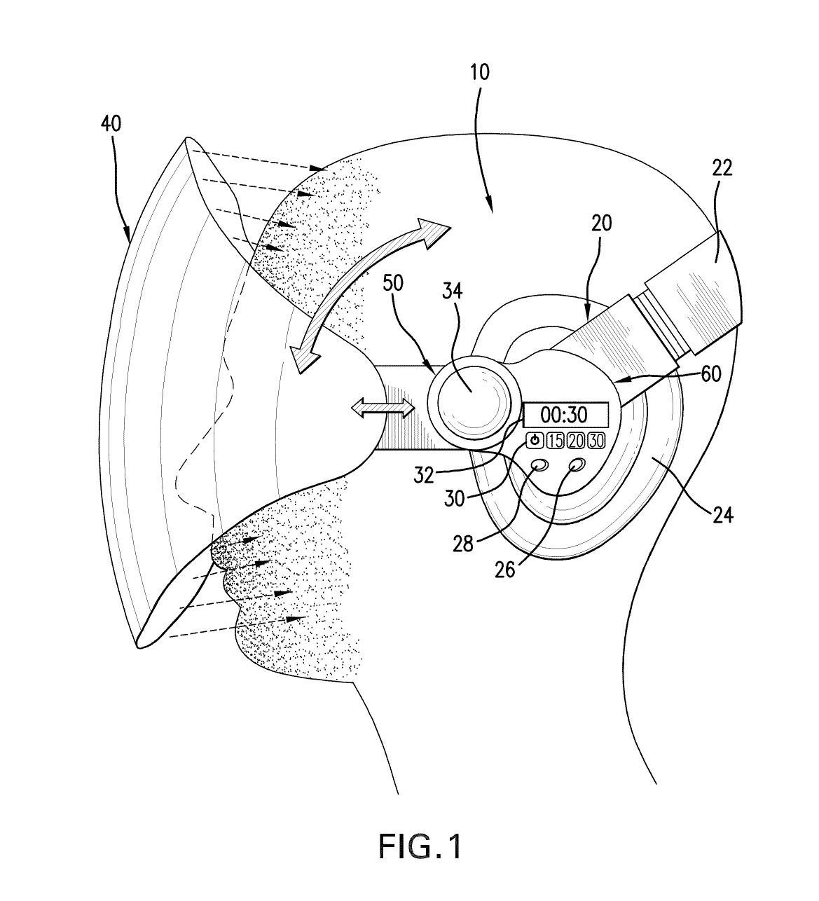

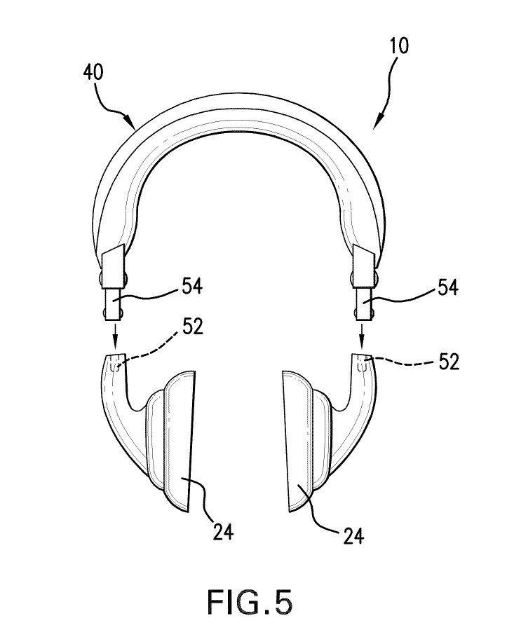

The embodiment shown in FIGS. 1-5 provides for interchangeable canopy bands or plates. Each interchangeable canopy band 40 removably attaches to a supporting head unit that is meant to be worn on a user's head. In the embodiment shown in FIG. 1, the head unit is a headset unit 20. The canopy band 40 is supported by the headset unit such that light is directly emitted toward the user's face. The headset unit 20 includes a detachable, adjustable head support band 22, which can be adjusted for snuggly fitting on the user's head and is necessary to prevent the headphones from slipping. The headset unit 20 further includes two audio headphones 24 on opposite sides of the adjustable head support band 22, which are adapted to come in contact with the user's ears when the phototherapy apparatus 10 is properly worn on the user's head. An audio input 28 is located on the headset unit 20 and communicates with the two audio headphones 24, allowing the user to listen to an audio feed from any general audio device, such as an iPod. An LCD timer and function display system 32 is located on the headset unit 20, which displays a countdown timer and user functions, such as output wavelength. An input for a rechargeable battery system 26 is also located on the headset unit 20.

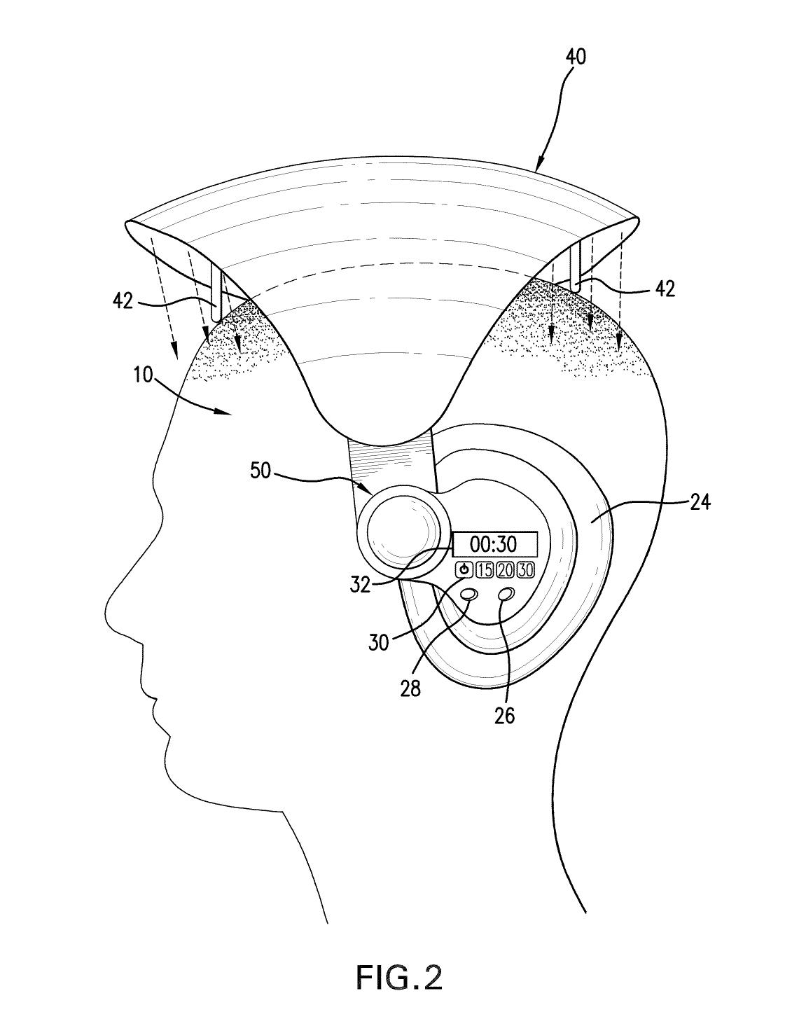

FIG. 2 shows a further embodiment of the phototherapy apparatus 10 wherein the canopy band 40 is rotated such that light is directed toward the top of the user's head (scalp). This second embodiment is particularly intended for treatment of hair loss, scalp and hair wellness, which requires light emitted within a range (628 nm-694 nm) of red wavelengths, but can also be used to treat other skin-related conditions that are present on the user's scalp. Further illustrated in FIG. 2 is the inclusion of spacing columns 42 located on both the frontal and posterior portions of the canopy band 40, allowing for accurately maintained placement of the canopy band 40 relative to the user's head.

As illustrated in both FIGS. 1 and 2, there is a headphone proprietary pivot point 34 connected to the headphones 24, which allows for rotational movement of the canopy band 40 relative to the user's head, and consequently, complete scalp and facial coverage by the canopy band 40.

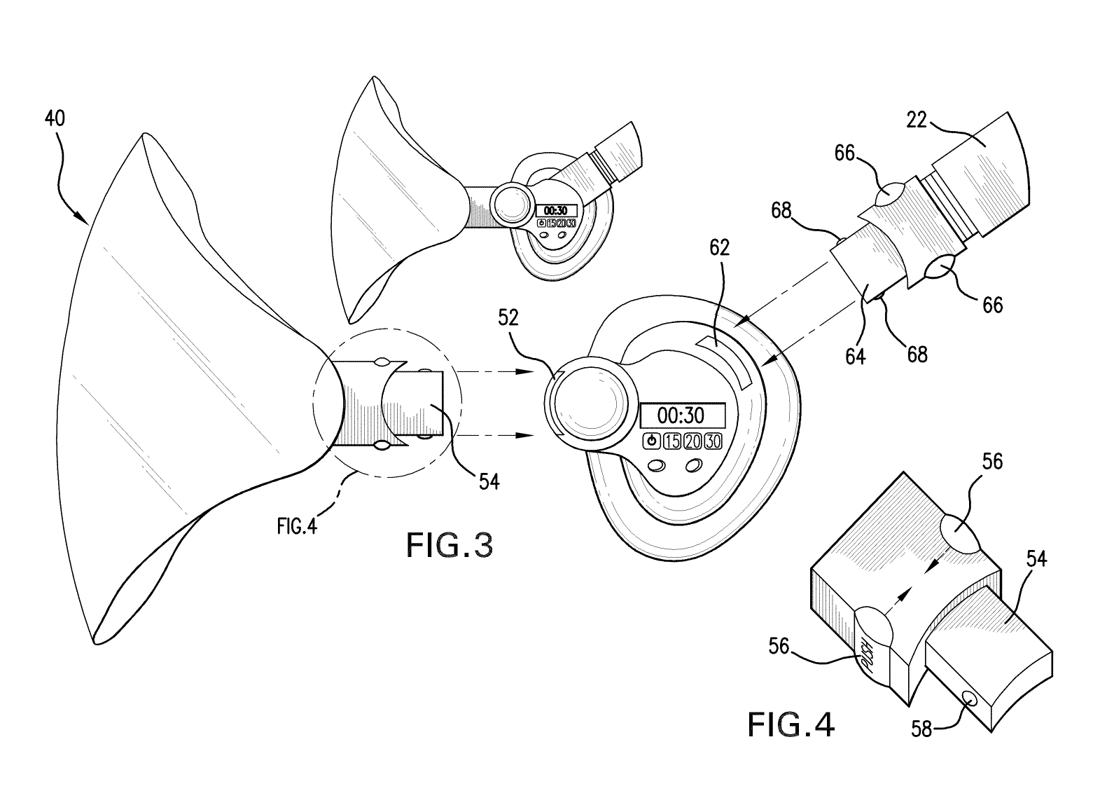

As shown in FIGS. 3-5, on each side of the headset unit 20 is a female component 52 of a releasable securing mechanism 50. On opposite sides of each canopy band 40 is a male component 54 of the releasable securing mechanism 50. Each male component 54 has a release button 56 and a release mechanism 58. In operation, the male component 54 snaps into the female component 52 and securely fixes the canopy band 40 with the headset unit 20. In order to separate the canopy band 40 from the headset unit 20, the user must squeeze together the opposite ends of the release button 56, which will unhinge the release mechanism 58 and allow separation of the male component 54 from the female component 52.

Further illustrated in FIGS. 3 and 4 is the head support band securing mechanism 60, which helps support the phototherapy apparatus 10 upon the user's head when required. The head support band securing mechanism 60 is comprised of dual female components 62 that are located on the headphones 24, and duel male components 64 that are located on the head support band 22. In operation, the male component 64 snaps into the female component 62 and securely fixes the headphones 24 with the head support band 22. In order to separate the headphones 24 from the head support band 22, the user must squeeze together the opposite ends of the release button 66, which will unhinge the release mechanism 68 and allow separation of the male component 64 from the female component 62.

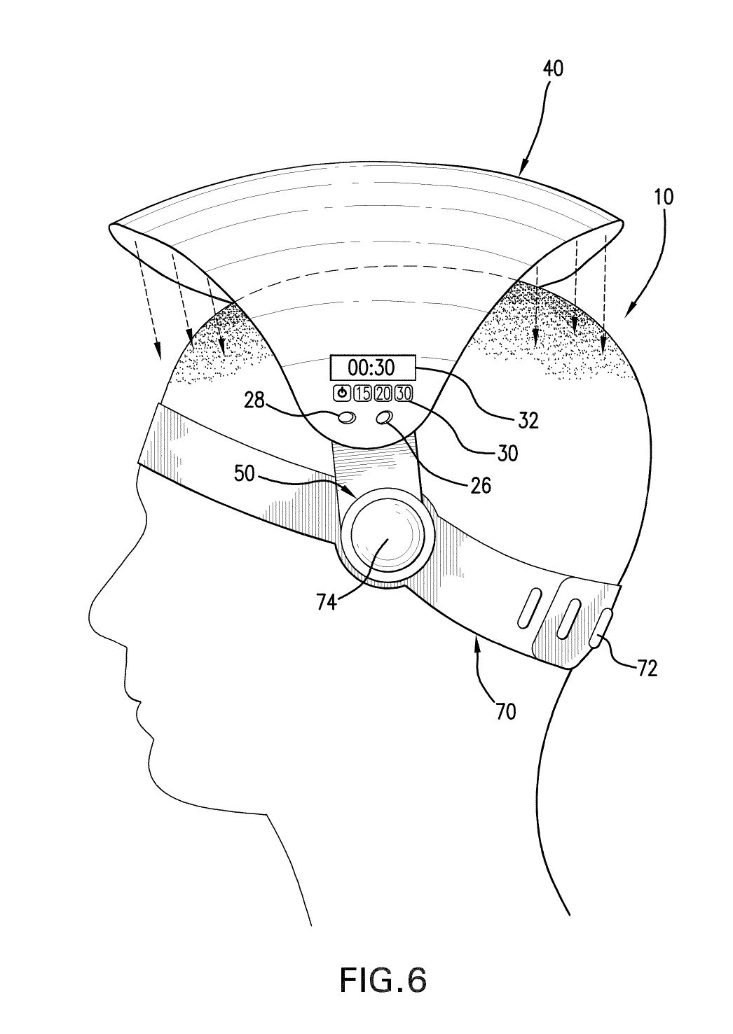

In another embodiment of the phototherapy apparatus 10, a canopy band 40 is supported by a headband unit 70, which is adapted to be worn on the user's head, as shown in FIG. 6. The canopy band 40 houses an array of light generating sources 102 on its inner facing side 100 that provides evenly distributed phototherapy treatment to the user's scalp for treatment of hair-related conditions.

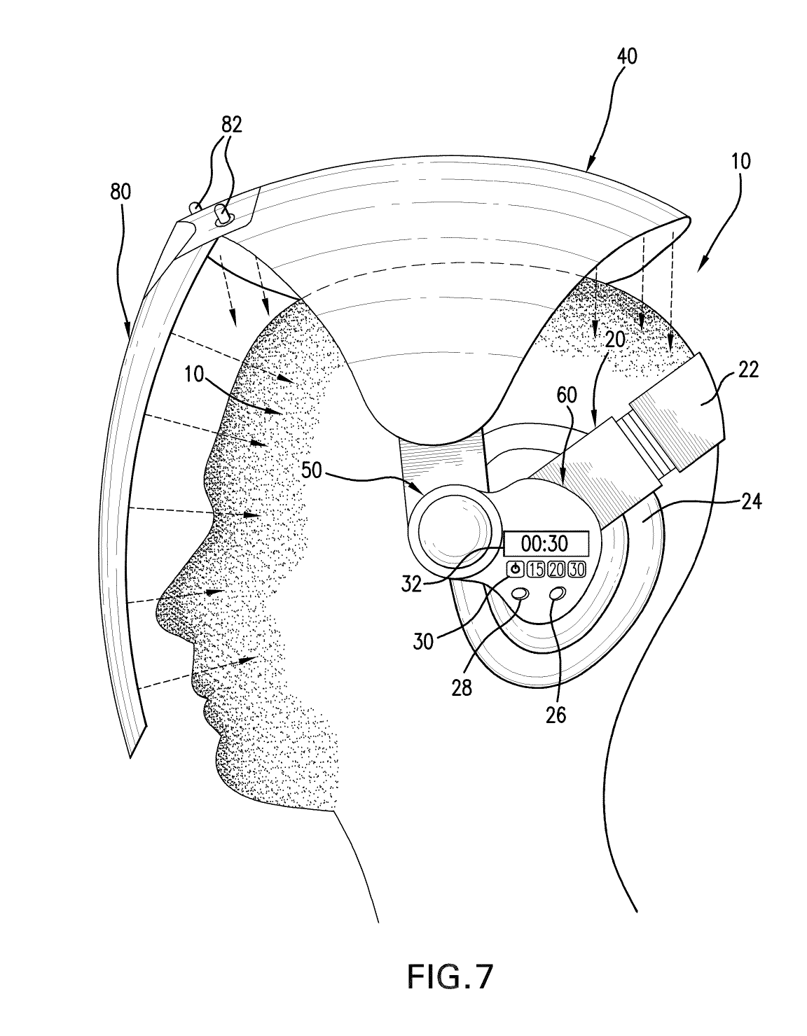

As illustrated in FIG. 7, the addition of a fixed or detachable face plate 80 connected to the canopy band 40 provides for a further embodiment of the phototherapy apparatus 10. The fixed or detachable face plate 80 houses an array of light generating sources 102 on its inner facing side, designed for providing evenly distributed phototherapy treatment to the user's face. This embodiment allows the user the option of treating both the scalp and face regions of the user's head, as the canopy band 40 in this embodiment is positionable in spaced, opposing relation to the scalp for treatment of hair-related conditions in the same manner as is provided in the second embodiment of the phototherapy apparatus 10.

As illustrated in both FIGS. 6 and 7, there is a headband proprietary pivot point 74 connected to the headband 70, which allows for rotational movement of the canopy band 40 relative to the user's head, and consequently, complete scalp and facial coverage by the canopy band 40.

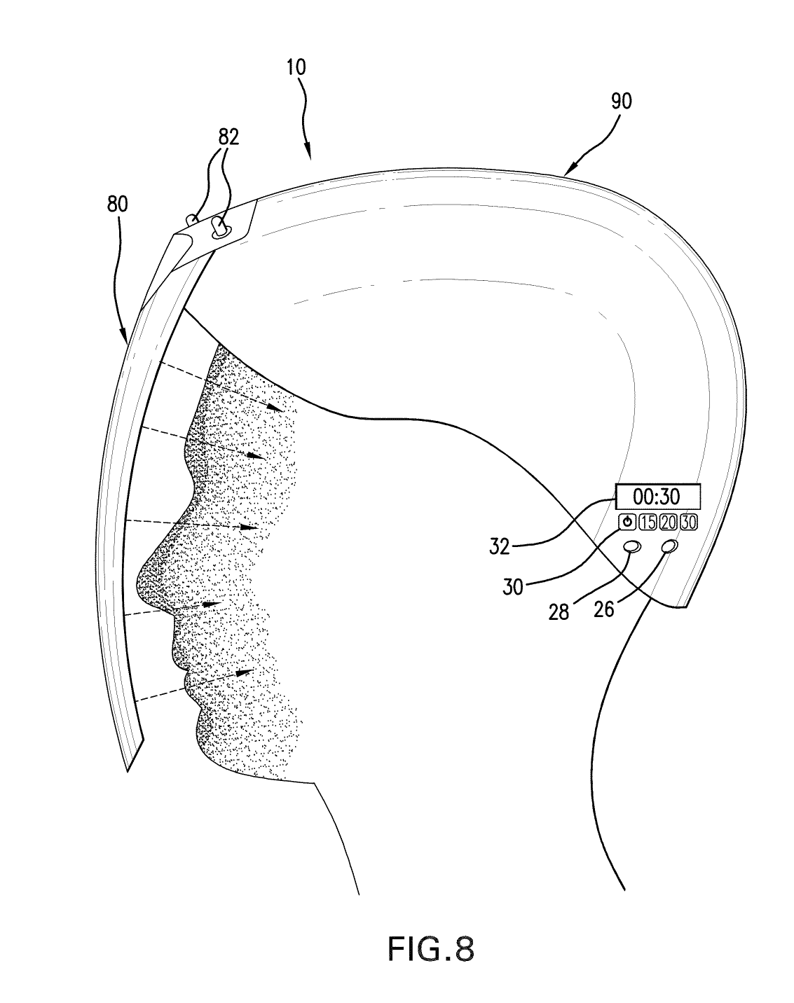

Another embodiment of the phototherapy apparatus 10 is shown in FIG. 8, which illustrates a helmet unit 90 adapted to be worn on a user's head. Attached to the helmet unit 90 is a fixed or detachable face plate 80, which houses an array of light generating sources 102 on its inner facing side, designed for providing evenly distributed phototherapy treatment to the user's face.

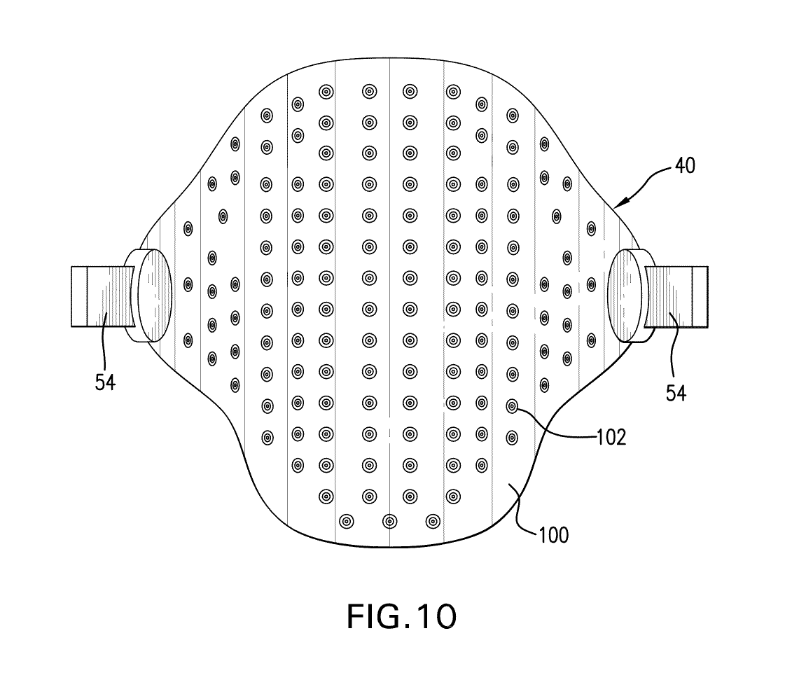

As illustrated in FIGS. 9 and 10, an array of light generating sources 102 are mounted on the inner facing side 100 of a canopy band 40 that is positionable in spaced, opposing relation to a select area of the user's head. As an alternative to the array of light generating sources 102 being mounted on the inner facing side 100 of the canopy band 40, the array of light generating sources 102 can be snapped into place on the inner facing side 100 of the canopy band 40. In a further embodiment, the array of light generating sources 102 are composed of multiple wavelength light generating sources 102 within a single canopy band 40, wherein certain light generating sources 102 emit light within one particular wavelength range, while other light generating sources 102 emit light within different wavelength ranges. The spread of light from each light generating source 102 widens as it leaves the aperture, creating an overlap that provides a uniform distribution and intensity of light with enhanced penetration depth control to regions of skin tissue on the user's head. Additionally, protective eye wear can be worn by the user when the phototherapy apparatus 10 is being used to treat particular skin-related conditions on the face.

In each of the embodiments shown, depending on the type of condition being treated, light emitted at a particular output wavelength range is required to sufficiently penetrate the skin tissue. For example, in treating inflammation, lesions, or canker sores, a range (628 nm-694 nm) of red wavelengths is preferable; in treating rosacea or wrinkling of the skin, a range (568 nm-590 nm) of yellow wavelengths is preferable; in treating acne, a range (405 nm-476 nm) of blue wavelengths is preferable; in treating age spots, sun damage, or hyperpigmentation, a range (514 nm-543 nm) of green wavelengths is preferable; and in stimulating the skin to produce collagen and elastin, a range (700-1090 nm) of infrared wavelengths is preferable. For treating hair loss, light generating sources with a 670 nm output wavelength will produce a penetration depth of approximately 2-8 mm for direct treatment of hair cells.

It is noted that in each of the embodiments of the phototherapy apparatus 10 shown and described above, electric power for energizing the array of light generating sources may be supplied by disposable or rechargeable batteries carried in the head unit 12 or hand held control device 110. Alternatively, the head unit may plug into a standard wall outlet (e.g., a 110 volt outlet) for supplying electric power to the light generating sources. Similarly, electric power for energizing the audio source and functions, as well as the LCD display 120 and control circuitry of the hand held device 110 can be supplied by disposable or rechargeable battery power or by plugging into a standard wall outlet.

While the invention has been shown and described in accordance with several preferred and practical embodiments thereof, it is recognized that departures from the instant disclosure of the invention are fully contemplated within the spirit and scope of the invention and such changes, variations and modifications of the present invention are not to be limited except as recited in the following claims as interpreted under the Doctrine of Equivalents.

What is claimed is:

1. A wearable hands-free apparatus for providing phototherapy treatment to a user, said apparatus comprising:
a head unit adapted to be worn on the user's head and including a pair of audio emitting earphones positionable on the user's ears;
at least one canopy band supported on said head unit, and said at least one canopy band including an inner facing side that is positionable in spaced, opposing relation to a select area of the user's head;
an array of light generating sources on said inner facing side of said at least one canopy band and being structured and disposed for producing a light pattern that is directed onto the select area of the user's head, and each of said light generating sources being further structured and disposed for emitting light within a wavelength range according to a particular condition being treated by phototherapy;
at least one control on said apparatus for controlling operation of said array of light generating sources.

2. The apparatus as recited in claim 1 wherein said light generating sources are light emitting diodes (LEDs).

3. The apparatus as recited in claim 1 wherein said light generating sources are laser diodes.

4. The apparatus as recited in claim 1 wherein said light generating sources are intense pulse lights (IPLs).

5. The apparatus as recited in claim 1 wherein said light generating sources are infrared lights.

6. The apparatus as recited in claim 1 wherein said head unit includes an LCD timer and function display system.

7. The apparatus as recited in claim 6, further comprising a face plate structured and disposed for connecting to each of said at least one canopy band, and said face plate being further structured and disposed for housing said array of light generating sources.

8. The apparatus as recited in claim 1 wherein said at least one control is a hand held device with an LCD display, and said hand held device including a programmable memory for storing an algorithm that controls the timing and pulse rate of said array of light generating sources in accordance with a plurality of specific phototherapy treatments.

9. A wearable hands-free phototherapy apparatus for treatment of a region of skin cells and layers of a user's dermis, comprising:
a head unit adapted to be worn on the user's head;
at least one canopy band removably attachable to said head unit and including an inner facing side that is positionable in spaced, opposing relation to a select area of the user's face and scalp;
a pivot point on said apparatus structured and disposed for allowing rotation of each said at least one canopy band to a plurality of adjusted positions relative to the user's head;
an array of light generating sources on said inner facing side of said at least one canopy band and being structured and disposed for producing a light pattern that is directed onto the select area of the user's face and scalp, and each of said light generating sources being further structured and disposed for emitting light within a selected wavelength range according to a particular condition being treated by phototherapy; and
at least one control on said apparatus for controlling operation of said array of light generating sources.

10. The apparatus as recited in claim 9 wherein said light generating sources are light emitting diodes (LEDs).

11. The apparatus as recited in claim 9 wherein said light generating sources are laser diodes.

12. The apparatus as recited in claim 9 wherein said light generating sources are intense pulse lights (IPLs).

13. The apparatus as recited in claim 9 wherein said light generating sources are infrared lights.

14. The apparatus as recited in claim 9 wherein said head unit includes an LCD timer and function display system.

15. The apparatus as recited in claim 14, further comprising a face plate structured and disposed for connecting to each of said at least one canopy band, and said face plate being further structured and disposed for housing said array of light generating sources.

16. A wearable hands-free phototherapy apparatus for treatment of a region of skin cells and layers of a user's dermis, comprising:
a head unit adapted to be worn on the user's head;
a plurality of canopy bands removably attachable to said head unit and including an inner facing side that is positionable in spaced, opposing relation to a select area of the user's face and scalp;
a pivot point on said apparatus structured and disposed for allowing rotation of each said plurality of canopy bands to a plurality of adjusted positions relative to the user's head;
an array of light generating sources on said inner facing side of said plurality of canopy bands and being structured and disposed for producing a light pattern that is directed onto the select area of the user's face and scalp, and each of said light generating sources being further structured and disposed for emitting light within a selected wavelength range according to a particular condition being treated by phototherapy;
a first of said plurality of canopy bands having said array of light generating sources adapted for emitting light within the selected wavelength ranges of 405 nm-476 nm;
a second of said plurality of canopy bands having said array of light generating sources adapted for emitting light within the selected wavelength ranges of 514 nm-543 nm;
a third of said plurality of canopy bands having said array of light generating sources adapted for emitting light within the selected wavelength ranges of 568 nm-590 nm;
a fourth of said plurality of canopy bands having said array of light generating sources adapted for emitting light within the selected wavelength ranges of 628 nm-694 nm;
a fifth of said plurality of canopy bands having said array of light generating sources adapted for emitting light within the selected wavelength ranges of 700 nm-1090 nm; and
at least one control on said apparatus for controlling operation of said array of light generating sources.

17. The apparatus as recited in claim 16 wherein said light generating sources are light emitting diodes (LEDs).

18. The apparatus as recited in claim 16 wherein said light generating sources are laser diodes.

19. The apparatus as recited in claim 16 wherein said light generating sources are intense pulse lights (IPLs).

20. The apparatus as recited in claim 16 wherein said light generating sources are infrared lights.

21. The apparatus as recited in claim 16 wherein said head unit includes an LCD timer and function display system.

22. The apparatus as recited in claim 21, further comprising a face plate structured and disposed for connecting to each of said plurality of canopy bands, and said face plate being further structured and disposed for housing said array of light generating sources.

23. A wearable hands-free phototherapy apparatus for treatment of a region of skin cells comprising:
a head unit adapted to be worn on a user's head;
at least one canopy band attached to said head unit and including an inner facing side that is positionable in spaced, opposing relation to a select area of the user's scalp;
a pivot point on said apparatus structured and disposed for allowing rotation of each said at least one canopy band to a plurality of adjusted positions;
an array of light generating sources on said inner facing side of said at least one canopy band and being structured and disposed for producing a light pattern that is directed onto the select area of the user's scalp, and each of said light generating sources being further structured and disposed for emitting light within a selected wavelength range; and
at least one control for controlling operation of said array of light generating sources.

24. The apparatus as recited in claim 23, wherein said light generating sources are light emitting diodes (LEDs).

25. The apparatus as recited in claim 23, wherein said light generating sources are laser diodes.

26. The apparatus as recited in claim 23, wherein said light generating sources are infrared lights.

27. The apparatus as recited in claim 23, wherein the array of light generating sources comprises light generating sources that emit light at a first wavelength range and light generating sources that emit light as a second wavelength range.
